(19) United States
(12) Patent Application Publication
(10) Pub. No.: US 2009/0215855 A1
(43) Pub. Date: Aug. 27, 2009

(54) New Crystalline Atorvastatin Hemicalcium Salt Polymorph Form

(76) Inventors: Jozsef Barkoczy, Budapest (HU); Norbert Nemeth, Sopran (HU); Gyorgy Ruzsics, Hogyesz (HU); Judit Cselenyak, Budapest (HU)

Correspondence Address: K.F. ROSS P.C., 5683 RIVERDALE AVENUE, SUITE 203 BOX 900, BRONX, NY, 10471-0900, US

(21) Appl. No.: 11/887,890

(22) PCT Filed: Apr. 7, 2006
(86) PCT No.: PCT/HU2006/000026

§ 371 Date: Dec. 11, 2008

(30) Foreign Application Priority Data
Apr. 8, 2005 (HU) P 05 00370
Feb. 14, 2006 (HU) P 06 00120

(51) Int. Cl. A61K 31/40; C07D 207/34

(52) U.S. Cl. 514/423; 548/537

(57) ABSTRACT

The present invention relates to a new crystalline polymorph B-52 form of atorvastatin hemicalcium salt [A[(3R,5R)-7-[3-phenyl-4-[(phenyl carbamoyl]-2-(4-fluorophenyl)-5-(1-methylethyl)-1H-pyrrole-1-yl]-3,5-dihydroxy-heptanoic acid calcium salt (2:1)], medicinal preparations containing the new polymorph form, process for the preparation thereof and the use of the new polymorph form for the preparation of medicinal products.

### FIELD OF THE INVENTION

The present invention relates to a new crystalline polymorph form of atorvastatin hemicalcium salt [(3R,5R)-7-[3-phenyl-4-[(phenylcarbamoyl)]-2-(4-fluorophenyl)-5-(1-methylethyl)-1H-pyrrole-1-yl]-3,5-dihydroxy-heptanoic acid calcium salt (2:1)] of the Formula (I),

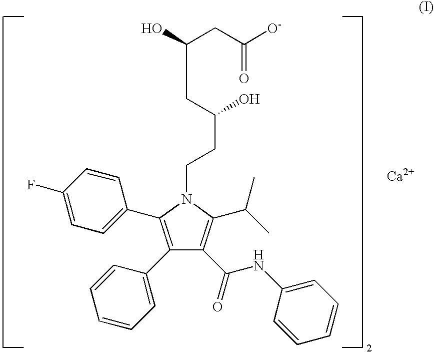

medicinal preparations containing the new polymorph form, process for the preparation thereof and the use of the new polymorph form for the preparation of medicinal products.

### TECHNICAL BACKGROUND OF THE INVENTION

Atorvastatin hemicalcium salt [(3R,5R)-7-[3-phenyl-4-[(phenylcarbamoyl)-2-(4-fluorophenyl)-5-(1-methylethyl)-1H-pyrrole-1-yl]-3,5-dihydroxy-heptanoic acid calcium salt (2:1)] of the Formula (I), which is designated in the subsequent part of the present application as atorvastatin hemicalcium salt, is an inhibitor of the enzyme 3-hydroxy-3-methyl-glutaryl-coenzime A reductase present in the liver. Said enzyme plays distinguished role in the biosynthesis of cholesterol. Due to this effect, atorvastatin hemicalcium salt of the Formula (I) can be very effectively used as pharmaceutical active ingredient for decreasing blood lipid- and cholesterol concentration.

Several processes for the preparation of atorvastatin hemicalcium salt have been disclosed. Said processes comprise the subject matter of European Patents No. 330172, 409281, 448552, 553213 and 687263. In the subsequent part of the present application, atorvastatin hamicalcium salt prepared according to European Patent No. 409281 is referred as crude atorvastatin hemicalcium salt.

Processes for the preparation of crystalline polymorph form I, II and IV of atorvastatin hemicalcium salt have been disclosed in European Patent No. 848705. According to said patent, polymorph I crystalline form of atorvastatin hemicalcium salt is obtained either by seeding the solution of atorvastatin hemicalcium salt with seed crystals of polymorph I form atorvastatin hemicalcium salt or by suspending and stirring the mixture of amorphous and crystalline form I atorvastatin hemicalcium salt in 200-fold volume of methanol-water mixture at 40° C. and crystallizing polymorph form I atorvastatin hemicalcium salt during the period of 17 hours.

Crystalline polymorph form II atorvastatin hemicalcium salt is prepared from the mixture of amorphous atorvastatin hemicalcium salt and crystalline polymorph form I atorvastatin hemicalcium salt by suspending the starting material in 20-fold amount of 3:2(v/v) mixture of methanol and water and stirring the resulting suspension for 3 days.

Crystalline polymorph form IV atorvastatin hemicalcium can be prepared from atorvastatin lactone. The lacton is converted into hemicalcium salt in solution, the aqueous mixture is heated at least to 65-70° C. for 5 minutes, and cooled to 55-65° C. The crystals are filtered, stirred in methanol at 55-65° C., the suspension is cooled to 25-30° C. and the polymorph form IV of atorvastatin hemicalcium is filtered off.

Crystalline atorvastatin hemicalcium polymorph form III has been disclosed in European Patent No. 848704.

Beyond the above mentioned crystalline forms, numerous further crystalline polymorph forms of atorvastatin hemicalcium salt have been prepared. Such crystalline forms are disclosed in European Patent Application Nos. 1235799, 1332130, 1341785, 1363621, 1425287, 1465865, 1480950, 1423364, 1414796, 1472220 and in the International Patent Application No. WO 02/43732. Crystalline forms of atorvastatin hemicalcium salt of the Formula (I) additionally to atorvastatin hemicalcium contain 1-9 mol water bound in the crystalline lattice, calculated on the basis of the molar amount of atorvastatin hemicalcium salt.

According to the state of the art, the amorphous atorvastatin hemicalcium salt is also known and can be prepared by the processes disclosed in European Patent No. 839132 or in International Patent Application No. WO 01/28999.

The common feature of all the above mentioned processes directed to the preparation of crystalline forms of atorvastatin hemicalcium salt resides in the fact that said processes utilize controlled crystallization, wherein the polarity of the solution containing atorvastatin hemicalcium salt is decreased by the addition of apolar or less polar solvent, until atorvastatin hemicalcium is obtained in crystalline form, or solid amorphous or crystalline atorvastatin hemicalcium salt is stirred in a solvent or a solvent mixture until a crystalline form of atorvastatin hemicalcium salt different from that of the starting material is obtained.

In the conventional crystallization process, however, solvent is added to the substance to be recrystallized and the resulting suspension is heated until dissolution. The solution thus obtained is filtered to remove mechanical and less soluble impurities, which is essential to achieve the required degree of purity. Filtration plays important role in the development of uniform crystalline form, since externally introduced nuclei of crystallization can sometimes be removed only this way. Subsequent to filtration, the filtrate is cooled, which results in the crystallization of the dissolved substance. In order to enhance crystallization, the suspension is cooled further with ice water or another coolant and the resulting crystalline product is filtered.

In the International Patent Application No. WO 01/28999, a process for the preparation of amorphous atorvastatin hemicalcium has been disclosed. Said process comprises precipitation of atorvastatin hemicalcium from a protic solvent, for example, from 2-propanol. During the industrial application of the process, the objective of the inventors was to achieve better yield. According to the general experience in similar cases, increasing the amount of the less polar or aprotic solvent results in increasing yield. The greatest applicable amount of the aprotic solvent is limited by the purity requirements of the product. Furthermore, when atorvastatin hemicalcium salt was precipitated as an amorphous solid from protic solvents, one skilled in the art would expect that using a mixture of a protic solvent and a less polar aprotic solvent will provide favourable conditions for the formation of an amorphous product.

Similar conclusion was reached by the inventors of European Patent Application No. 1 237 864, who dissolved atorvastatin hemicalcium in the protic solvent methanol, concentrated the solution by partly evaporating the solvent and mixed the resulting solution with diethylether, which resulted in the formation of amorphous atorvastatin hemicalcium salt.

### SUMMARY OF THE INVENTION

The technical problem to be solved by our research was to provide crystalline atorvastatin hemicalcium salt in a high purity, stable, uniform, non-hygroscopic form suitable for the use in the preparation of medicinal products.

The above objective has been solved according to the present invention.

Surprisingly, we have found that by dissolving crude, amorphous or crystalline atorvastatin hemicalcium salt, solvates or mixtures thereof in a protic solvent or in a mixture comprising one or more protic solvent and if desired, an aprotic solvent, optionally by heating with subsequent filtering or optional incubation at a constant temperature, or if desired, addition of a seeding crystal, a new crystalline polymorph form of atorvastatin hemicalcium salt can be obtained upon cooling.

### DETAILED DESCRIPTION OF THE INVENTION

In the present patent application, the meaning of the expression “solvate” is a salt which contains a solvent residue which can not be removed under the conditions of vacuum drying.

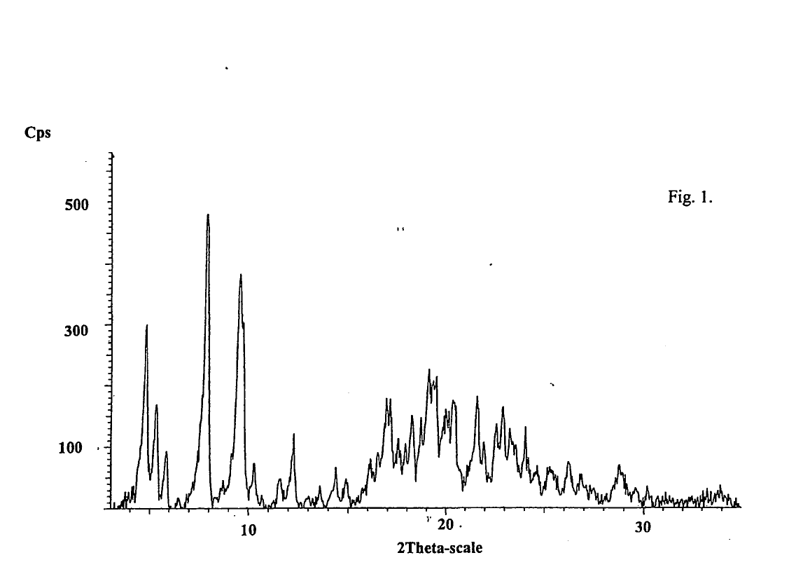

According to the first aspect of our invention, there is provided a new crystalline polymorph B-52 form of atorvastatin hemicalcium salt [(βR,δR)-2-(4-fluorophenyl)-β,δ-dihydroxy-5-(1-methylethyl)-3-phenyl-4-{(phenylamino)-carbpnyl}-1H-pyrrole-1-heptanoic acid hemicalcium salt] of the Formula (I) and its solvates, which correspond to the X-ray diffraction data demonstrated in Table 1 and in FIG. 1.

Crystalline polymorph form B-52 of atorvastatin hemicalcium is a high purity, stable, non-hygroscopic substance, suitable for the use in the preparation of medicinal products, having advantageous properties for the use in pharmaceutical technology.

X-ray diffraction data of the crystalline form B-52 atorvastatin hemicalcium polymorph form have been determined under the following experimental conditions.

| | |
|---|---|
| Instrument: | Bruker D8 Advanced Powder Diffractometer |
| Radiation: | CuK_{α1} (λ = 1,54060 Å), CuK_{α2} (λ = 1,54439 Å) |
| Voltage | 40 kV |
| Plate current | 30 mA |
| Accessories | Göbel mirror |
| | Soller slit |

During the determination of X-ray diffraction data, the reference substance SRM 640c (Silicon Powder Lot. No. H-375) was used. Measurements were carried out continuously in the diffraction angle range of 4 to 30° (2Θ) in 0.04° steps. The X-ray analysis was carried out on smooth samples stored at room temperature without milling. Measurements were made at room temperature.

X-ray diffraction pattern of the atorvastatin hemicalcium polymorph B-52 form according to our invention is demonstrated in FIG. 1. Data of the X-ray diffraction signals are listed in Table 1.

Data of FIG. 1 and Table 1 are to be interpreted in the present application by taking into account the repeatability of X-ray diffraction measurements known according to the state of the art. It is known from the state of the art, for example, from pharmacopoeias that the repeatability of the measurement of X-ray diffraction angles is approximately ±0.2°. Furthermore it is known according to the state of the art that the intensity of the X-ray diffraction peaks is significantly influenced by the sample condition or the sample preparation method. In the present patent application, polymorph forms of atorvastatin hemicalcium salt are considered identical if their measured X-ray diffraction angles correspond to each other within the above mentioned repeatability and the relative intensities determined at a given diffraction angle do not differ more than about ±20 relative percent.

According to the second aspect of the present invention, there is provided a process for the preparation of atorvastatin hemicalcium salt of the Formula (I) in the B-52 polymorph form, which comprises dissolving crude, amorphous or crystalline atorvastatin hemicalcium salt or solvates or mixtures thereof in a protic solvent or in a protic solvent mixture, which may also contain an aprotic solvent, filtering the solution, optionally seeding the solution with crystalline B-52 form atorvastatin hemicalcium salt seeding crystals, cooling and stirring the mixture at room temperature and filtering, washing and drying the crystals of the atorvastatin hemicalcium polymorph B-52 form thus obtained.

As protic solvent, an aliphatic alcohol comprising 1-4 carbon atoms or the mixture of an aliphatic alcohol comprising 1-4 carbon atoms and water, wherein the proportion of water in said mixture is less than 5 volume % can be used.

As an aprotic solvent, apolar solvents, such as an alkane or cycloalkane comprising 5-8 carbon atoms, as a less polar solvent, for example, a dialkyl-ether comprising 4-8 carbon atoms or an aliphatic ester or ketone comprising 3-8 carbon atoms, can be used.

The weight of the solvent or the solvent mixture used can be chosen according to the properties of the solvent or the solvent mixture between 2-fold and 50-fold weight of the starting substance.

If necessary, heating is applied to facilitate the dissolution of the starting atorvastatin hemicalcium salt.

After filtering the solution containing atorvastatin calcium, the mixture is cooled to room temperature, optionally seeding crystals are added and the mixture is stirred at room temperature for 0.1 to 48 hours, preferably, for 4 to 20 hours. The crystals of atorvastatin hemicalcium salt polymorph B-52 form thus obtained are filtered off, washed and dried.

TABLE 1
X-ray diffraction data of atorvastatin
hemicalcium salt polymorph B-52 form

| Diffraction | | | Relative |
|---|---|---|---|
| Angle (2Θ) | d value | Intensity | Intensity |
| (°) | (Å) | (cps) | (%) |
| 4.485 | 19.6865 | 105 | 21.7 |
| 4.715 | 18.7267 | 298 | 61.9 |
| 5.270 | 16.7560 | 168 | 34.9 |
| 5.766 | 15.3161 | 91.6 | 19.0 |
| 7.806 | 11.3165 | 481 | 100 |
| 9.535 | 9.26799 | 383 | 79.7 |
| 10.245 | 8.62703 | 71.9 | 14.9 |
| 11.582 | 7.63438 | 46.4 | 9.6 |
| 12.177 | 7.26235 | 88.5 | 18.4 |
| 14.327 | 6.17731 | 56.5 | 11.7 |
| 16.097 | 5.50159 | 78.7 | 16.3 |
| 16.480 | 5.37470 | 87.6 | 18.2 |
| 16.911 | 5.23878 | 178 | 36.9 |
| 17.083 | 5.18634 | 163 | 33.8 |
| 17.542 | 5.05155 | 108 | 22.5 |
| 17.887 | 4.95502 | 104 | 21.6 |
| 18.243 | 4.85894 | 150 | 31.1 |
| 18.691 | 4.74367 | 144 | 30.0 |
| 19.093 | 4.64470 | 225 | 46.7 |
| 19.437 | 4.56314 | 212 | 44.1 |
| 19.983 | 4.43979 | 160 | 33.3 |
| 20.356 | 4.35922 | 172 | 35.7 |
| 21.562 | 4.11808 | 181 | 37.5 |
| 21.935 | 4.04884 | 101 | 21.1 |
| 22.595 | 3.93198 | 131 | 27.3 |
| 22.887 | 3.88247 | 163 | 33.9 |
| 23.284 | 3.81716 | 126 | 26.1 |
| 24.085 | 3.69203 | 130 | 27.1 |
| 24.597 | 3.61630 | 67.1 | 13.9 |
| 25.297 | 3.51784 | 66.0 | 13.7 |
| 26.224 | 3.39554 | 74.2 | 15.4 |
| 26.846 | 31.31830 | 56.6 | 11.8 |
| 28.797 | 3.09776 | 66.9 | 13.9 |

According to a further aspect of the present invention, there are provided medicinal preparations comprising atorvastatin hemicalcium salt polymorph B-52 form and one or more pharmaceutically acceptable vehicle or auxiliary agent.

The medicinal preparations according to the present invention generally contain 0.1-95 weight %, advantageously 1-50 weight %, the most preferably 5-30 weight % active ingredient.

The medicinal preparations according to the present invention can be administered orally (for example, in the form of powders, tablets, coated tablets, capsules, microcapsules, dragees, solutions, emulsions etc.), parenterally (for example, as intravenous, intramuscular, subcutaneous or intraperitoneal injection solutions or as solution for infusion), rectally (e.g. in the form of suppositories), transdermally (e.g. as patches), as implants or topically (e.g. creams, ointments or patches). The solid, semisolid, soft or liquid medicinal preparation according to the present invention can be prepared according to processes known for the person skilled in the art.

Solid medicinal preparations suitable for oral administration containing the polymorph B-52 form of atorvastatin hemicalcium salt according to the present invention can contain vehicles or fillers (e.g. lactose, lactose monohydrate, glucose, starch, calcium phosphate, calcium carbonate, microcrystalline cellulose), binders (e.g. gelatine, sorbite, polyvinyl-pyrrolidone), disintegrants (e.g. croscarmellose, sodium carboxymethyl cellulose, crospovidone) tabletting aids (e.g. magnesium stearate, talc, polyethylene glycol, silica, silicon dioxide), pH adjusting auxiliary agents (e.g. citric acid, phosphoric acid, lactic acid and alkali metal or alkali earth metal salts thereof; alkali metal or alkali earth metal carbonates or hydrogencarbonates, amino acids or amino-derivatives of carbohydrates) or surfactants (e.g. sodium laurylsulphate). Solid medicinal preparations can contain coating agents, e.g. hydroxypropylcellulose, hydroxypropyl-methylcellulose, polyvinylalcohol, polyethylene glycol, acrylate polymers, titanium dioxide or iron-oxide.

Liquid pharmaceutical preparations containing atorvastatin hemicalcium salt in the polymorph B-52 form suitable for oral administration can be solutions, suspensions or emulsions and can contain suspending agents (e.g. gelatine, carboxymethylcellulose), emulsifying agents (e.g. sorbitane monooleate), solvents (e.g. water, oils, glycerol, propylene glycol, ethanol), pH adjusting agents (e.g. acetate, phosphate, citrate buffers) and stabilizing agents (e.g. methyl-4-hydroxy-benzoate).

Liquid pharmaceutical preparations containing atorvastatin hemicalcium salt polymorph B-52 form are usually sterile isotonic solutions, which contain besides the solvent pH adjusting and conserving agents.

Soft medicinal preparations containing atorvastatin hemicalcium polymorph B-52 form as active ingredient, e.g. suppositories contain said active ingredient homogeneously dispersed in the vehicle of said preparation (e.g. polyethylene glycol, cocoa butter).

Medicinal preparations containing the atorvastatin hemicalcium polymorph B-52 form as active ingredient can be prepared according to methods of pharmaceutical technology known from the state of the art. The active ingredient is mixed with solid or liquid vehicles and auxiliary agents and the mixture is brought to galenic form. Vehicles and auxiliary agents suitable for use in medicinal products have been disclosed in the literature (Remington's Pharmaceutical Sciences, Edition 18, Mack Publishing Co., Easton, USA, 1990).

Medicinal preparations according to the present invention contain atorvastatin hemicalcium polimorf B-52 form in a dosage unit form.

A further aspect of the present invention is the use of crystalline atorvastatin hemicalcium B-52 polymorf form for the manufacture of medicinal preparations suitable for the reduce elevated plasma cholesterol, low density lipoprotein cholesterol, apo-β-lipoprotein- or triglyceride concentration and the treatment of hypercholesterolemia, dysbetalipoproteinemia and dyslipidemia, which comprises mixing crystalline atorvastatin hemicalcium salt B-52 polymorph form with pharmaceutically suitable vehicles and auxiliary agents and bringing the mixture to galenic form.

According to a still further aspect of the present invention, there is provided a method for the reduction of elevated plasma cholesterol, low density lipoprotein cholesterol, apo-β-lipoprotein- and triglyceride level and treatment of hypercholesterolemia, dysbetalipoproteinemia and dyslipidemia in a patient in need of such treatment, which comprises administering said patient an effective dose of crystalline atorvastatin hemicalcium salt B-52 polymorph form.

Further details of the present invention are provided in the following examples without limiting the scope of protection to said examples.

### Example 1

### Preparation of crystalline (3R,5R)-3,5-dihydroxy-[3-phenyl-4-[(phenylamino)-carbonyl]-2-(4-fluorophenyl)-5-(1-methylethyl)-1H-pyrrole-1-yl]-heptanoic acid hemicalcium salt B-52 polymorph form from crude atorvastatin hemicalcium salt

17 g crude atorvastatin hemicalcium salt prepared according to the method disclosed in European Patent No. 409281 are dissolved in the mixture of 100 ml of methanol and 150 ml of hexane at the boiling temperature of the mixture, until a clear solution is obtained. The solution is filtered, cooled to room temperature and subsequently stirred at room temperature for 12 hours. The crystals thus produced are filtered, washed with hexane and diethylether and dried. Yield 13.7 g (83%).

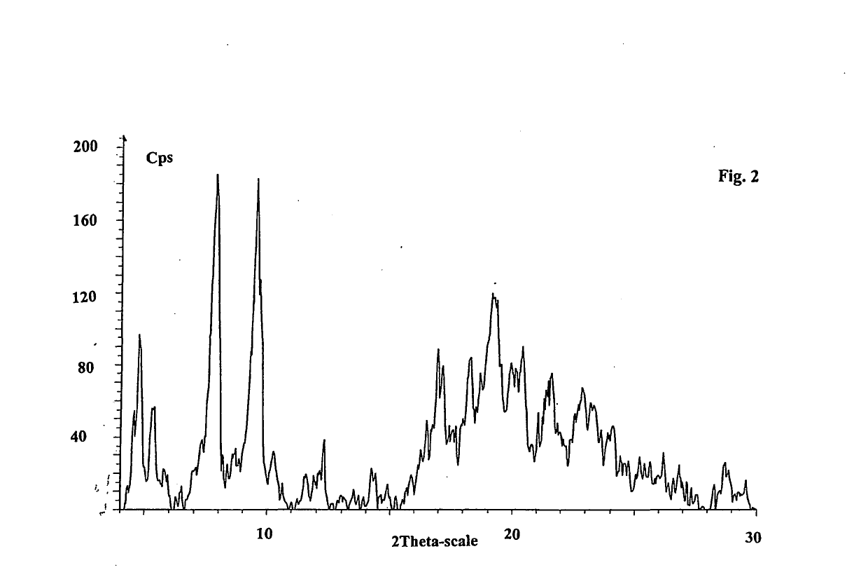

Results obtained by the X-ray diffraction analysis of the product are listed in Table 2. X-ray diffractogram of the product is shown in FIG. 2.

TABLE 2

| Diffraction | | | Relative |
|---|---|---|---|
| Angle (2Θ) | d value | Intensity | Intensity |
| (°) | (Å) | (cps) | (%) |
| 4.50 | 19.62575 | 52.6 | 28.4 |
| 4.70 | 18.81665 | 96.2 | 51.9 |
| 5.28 | 16.72994 | 56.4 | 30.4 |
| 5.75 | 15.35783 | 23.0 | 12.4 |
| 7.79 | 11.34293 | 185 | 100 |
| 9.50 | 9.30041 | 183 | 98.9 |
| 10.24 | 8.63159 | 31.6 | 17.1 |
| 11.52 | 7.67524 | 19.1 | 10.3 |
| 12.23 | 7.22997 | 38.2 | 20.6 |
| 14.23 | 6.21810 | 22.4 | 12.1 |
| 16.48 | 5.37343 | 49.7 | 26.8 |
| 16.90 | 5.24088 | 87.7 | 47.3 |
| 17.12 | 5.17615 | 79.9 | 43.1 |
| 18.23 | 4.86192 | 83.9 | 45.3 |
| 19.18 | 4.62341 | 118 | 63.7 |
| 19.91 | 4.45670 | 79.2 | 42.8 |
| 20.33 | 4.36386 | 84.3 | 45.5 |
| 21.49 | 4.13189 | 71.5 | 38.6 |
| 22.83 | 3.89231 | 67.0 | 36.1 |
| 23.27 | 3.82014 | 58.7 | 31.7 |
| 24.09 | 3.69128 | 45.4 | 24.5 |
| 28.72 | 3.10609 | 24.8 | 13.4 |

### Example 2

### Preparation of crystalline (3R,5R)-3,5-dihydroxy-[3-phenyl-4-[(phenylamino)-carbonyl]-2-(4-fluorophenyl)-5-(1-methylethyl)-1H-pyrrole-1-yl]-heptanoic acid hemicalcium salt B-52 polymorph form from amorphous atorvastatin hemicalcium salt

17 g amorphous atorvastatin hemicalcium salt prepared according to the disclosure of European Patent No. 1 235 799 is stirred until dissolution in the mixture of 100 ml of methanol and 150 ml of hexane at the boiling temperature of the mixture. Thus, a clear solution is obtained, which is filtered and cooled to room temperature and stirred at room temperature for further 12 hours. The precipitated crystals are filtered off, washed with hexane and diethylether and dried. Yield 13.7 g (83%).

### Example 3

### Preparation of crystalline (3R,5R)-3,5-dihydroxy-[3-phenyl-4-[(phenylamino)-carbonyl]-2-(4-fluorophenyl)-5-(1-methylethyl)-1H-pyrrole-1-yl]-heptanoic acid hemicalcium salt B-52 polymorph form from crude atorvastatin hemicalcium salt

17 g of crude atorvastatin hemicalcium salt prepared according to the disclosure of European Patent No. 409281 are dissolved in the mixture of 100 ml of methanol and 170 ml of diisopropylether at boiling temperature of the mixture, until a clear solution is obtained. The solution is filtered, cooled to room temperature and stirred at room temperature for further 12 hours. The crystals thus obtained were washed with hexane and diethylether and dried. Yield, 14.5 g (85%).

### Example 4

### Preparation of crystalline (3R,5R)-3,5-dihydroxy-[3-phenyl-[(phenylamino)-carbonyl]-2-(4-fluorophenyl)-5-(1-methylethyl)-1H-pyrrole-1-yl]-heptanoic acid hemicalcium salt B-52 polymorph form from crude atorvastatin hemicalcium salt

17 g of crude atorvastatin hemicalcium salt prepared according to the disclosure of European Patent No. 409281 was added to the mixture of 100 ml of methanol and 2 ml of water, heated to 40° C., and stirred for further 30 minutes at the same temperature. The mixture is filtered while hot and the filtrate is mixed with 1 g crystalline atorvastatin hemicalcium salt polymorph B-52 form. The obtained mixture is stirred at room temperature for 12 hours. The crystalline solids are filtered off, washed with methanol and dried. Yield, 13.6 g (80%).

### Example 5

### Preparation of crystalline (3R,5R)-3,5-dihydroxy-[3-phenyl-4-[(phenylamino)-carbonyl]-2-(4-fluorophenyl)-5-(1-methylethyl)-1H-pyrrole-1-yl]-heptanoic acid hemicalcium salt B-52 polymorph form from crude atorvastatin hemicalcium salt

17 g of crude atorvastatin hemicalcium salt obtained according to the processes described in European Patent No. 409281 are added to 100 ml methanol, the mixture is heated to boiling temperature and stirred at the same temperature for 30 minutes. The mixture is filtered while hot and the filtrate is mixed with 1 g of crystalline atorvastatin hemicalcium salt polymorph B-52 form. Subsequently, the mixture is stirred at room temperature for 12 hours. The crystalline solids thus obtained are filtered off, washed with methanol and dried. Yield, 13.6 g (80%). The X-ray diffractogram of the product is shown in FIG. 1. X-ray diffraction data of the product are given in Table 1.

### Example 6

### Preparation of crystalline (3R,5R)-3,5-dihydroxy-[3-phenyl-4-[(phenylamino)-carbonyl]-2-(4-fluorophenyl)-5-(1-methylethyl)-1H-pyrrole-1-yl]-heptanoic acid hemicalcium salt B-52 polymorph form from crystalline atorvastatin hemicalcium salt polymorph I form

17 g of atorvastatin hemicalcium salt polymorph I form prepared according to the process disclosed in European Patent No. 848705 are added to 100 ml of methanol, the mixture is heated to boiling and stirred at boiling temperature for 30 minutes. The mixture is filtered while hot and the filtrate are added to 1 g crystalline atorvastatin hemicalcium salt polymorph B-52 form.

The mixture is subsequently stirred at room temperature for 12 hours. The crystalline solid product is filtered off, washed with methanol and dried. Yield, 13.6 g (80%).

### Example 7

### Preparation of crystalline (3R,5R)-3,5-dihydroxy-[3-phenyl-4-[(phenylamino)-carbonyl]-2-(4-fluorophenyl)-5-(1-methylethyl)-1H-pyrrole-1-yl]-heptanoic acid hemicalcium salt B-52 polymorph form from crystalline atorvastatin hemicalcium salt polymorph I form

17 g of crude atorvastatin hemicalcium salt prepared according to the process disclosed in European Patent No. 409281 are added to the mixture of 50 ml of methanol and 125 ml of acetone, the mixture is heating to boiling and stirred at boiling temperature for further 30 minutes. The mixture is filtered while hot and 1 g crystalline atorvastatin hemicalcium salt polymorph B-52 form are added to the filtrate. The mixture is stirred at room temperature for further 12 hours. The crystalline solids are filtered off, washed with methanol and dried. Yield, 12.6 g (74%).

1. A crystalline atorvastatin hemicalcium salt [(βR,δR)-2-(4-fluorophenyl)-β,δ-dihydroxy-5-(1-methylethyl)-3-phenyl-4-{(phenylamino)-carbonyl}-1H-pyrrole-1-heptanoic acid hemicalcium salt] polymorph B-52 form and solvates thereof, having an X-ray diffractogram essentially identical to that depicted in FIG. 1 and which is characterized by the following X-ray diffraction data:

| Diffraction | | | Relative |
|---|---|---|---|
| Angle (2Θ) | d value | Intensity | Intensity |
| (°) | (A) | (cps) | (%) |
| 4.485 | 19.6865 | 105 | 21.7 |
| 4.715 | 18.7267 | 298 | 61.9 |
| 5.270 | 16.7560 | 168 | 34.9 |
| 5.766 | 15.3161 | 91.6 | 19.0 |
| 7.806 | 11.3165 | 481 | 100 |
| 9.535 | 9.26799 | 383 | 79.7 |
| 10.245 | 8.62703 | 71.9 | 14.9 |
| 11.582 | 7.63438 | 46.4 | 9.6 |
| 12.177 | 7.26235 | 88.5 | 18.4 |
| 14.327 | 6.17731 | 56.5 | 11.7 |
| 16.097 | 5.50159 | 78.7 | 16.3 |
| 16.480 | 5.37470 | 87.6 | 18.2 |
| 16.911 | 5.23878 | 178 | 36.9 |
| 17.083 | 5.18634 | 163 | 33.8 |
| 17.542 | 5.05155 | 108 | 22.5 |
| 17.887 | 4.95502 | 104 | 21.6 |
| 18.243 | 4.85894 | 150 | 31.1 |
| 18.691 | 4.74367 | 144 | 30.0 |
| 19.093 | 4.64470 | 225 | 46.7 |
| 19.437 | 4.56314 | 212 | 44.1 |
| 19.983 | 4.43979 | 160 | 33.3 |
| 20.356 | 4.35922 | 172 | 35.7 |
| 21.562 | 4.11808 | 181 | 37.5 |
| 21.935 | 4.04884 | 101 | 21.1 |
| 22.595 | 3.93198 | 131 | 27.3 |
| 22.887 | 3.88247 | 163 | 33.9 |
| 23.284 | 3.81716 | 126 | 26.1 |
| 24.085 | 3.69203 | 130 | 27.1 |
| 24.597 | 3.61630 | 67.1 | 13.9 |
| 25.297 | 3.51784 | 66.0 | 13.7 |
| 26.224 | 3.39554 | 74.2 | 15.4 |
| 26.846 | 3.31830 | 56.6 | 11.8 |
| 28.797 | 3.09776 | 66.9 | 13.9 |

2. The atorvastatin hemicalcium salt [βR,δR)-2-(4-fluorophenyl)-β,δ-dihydroxy-5-(1-methylethyl)-3-phenyl-4-{(phenylamino)-carbonyl}-1H-pyrrole-1-heptanoic acid hemicalcium salt] and solvates thereof comprising crystalline atorvastatin hemicalcium salt polymorph B-52 form according to claim 1 in admixture with amorphous atorvastatin hemicalcium salt or any crystalline atorvastatin hemicalcium salt different from B-52 polymorph form in its crystalline form.

3. The process for the preparation of crystalline atorvastatin hemicalcium polymorph B-52 form according to claim 1, which comprises dissolving crude, amorphous or crystalline atorvastatin hemicalcium salt, solvates thereof or mixtures thereof in a protic solvent or in a protic solvent mixture which can optionally contain an aprotic solvent, filtering, optionally seeding the clear solution with crystals of atorvastatin hemicalcium salt polymorph B-52 foam, cooling the mixture to room temperature, optionally incubating said mixture at constant temperature and separating the crystalline atorvastatin hemicalcium salt polymorph B-52 foam.

4. The process for preparation of crystalline atorvastatin hemicalcium polymorph B-52 form according to claim 1, which comprises dissolving crude atorvastatin hemicalcium salt, or its solvate in a protic solvent or in a protic solvent mixture which can optionally contain an aprotic solvent, filtering, optionally seeding the clear solution with crystals of atorvastatin hemicalcium salt polymorph B-52 form, cooling the mixture to room temperature, optionally incubating said mixture at constant temperature and separating the crystalline atorvastatin hemicalcium salt polymorph B-52 form.

5. The process for preparation of crystalline atorvastatin hemicalcium polymorph B-52 form according to claim 1, which comprises dissolving amorphous atorvastatin hemicalcium salt or its solvate in a protic solvent or in a protic solvent mixture which can optionally contain an aprotic solvent, filtering, optionally seeding the clear solution with crystals of atorvastatin hemicalcium salt polymorph B-52 form, cooling the mixture to room temperature, optionally incubating said mixture at constant temperature and separating the crystalline atorvastatin hemicalcium salt polymorph B-52 form.

6. The process for preparation of crystalline atorvastatin hemicalcium polymorph B-52 form according to claim 1, which comprises dissolving crystalline atorvastatin hemicalcium salt or its solvate in a protic solvent or in a protic solvent mixture which can optionally contain an aprotic solvent, filtering and optionally seeding the clear solution with crystals of atorvastatin hemicalcium salt polymorph B-52 form, cooling the mixture to room temperature, optionally incubating said mixture at constant temperature and separating the crystalline atorvastatin hemicalcium salt polymorph B-52 form.

7. The process according to claim 1, which comprises using an alcohol comprising 1 to 4 carbon atoms or water or mixtures thereof as a protic solvent.

8. The process according to claim 1, which comprises using an apolar solvent, e.g. an alkane or cycloalkane comprising 5-8 carbon atoms or a less polar solvent, such as a dialkyl ether comprising 4 to 8 carbon atoms or a dipolar aprotic solvent, such as and ester or ketone comprising 3 to 8 carbon atoms as aprotic solvent.

9. The process according to claim 1, which comprises using methanol optionally containing water as solvent.

10. The process according to claim 1, which comprises using a mixture of methanol and hexane as solvent.

11. The process according to claim 1, which comprises using a mixture of methanol and diisopropyl ether as solvent.

12. The process according to claim 1, which comprises using a mixture of methanol and acetone as solvent.

13. The process according to claim 1, which comprises using a solvent mixture containing 25-100 volume % protic solvent.

14. Crystalline atorvastatin hemicalcium salt [(δR,δR)-2-(4-fluorophenyl)-β,δ-dihydroxy-5-(1-methylethyl)-3-phenyl-4-{(phenylamino)-carbonyl}-1H-pyrrole-1-heptanoic acid hemicalcium salt] polymorph B-52 form and solvates thereof characterized by the X-ray diffractogram shown in FIG. 1 and X-ray diffraction data listed below,

| Diffraction | | | Relative |
|---|---|---|---|
| Angle (2Θ) | d value | Intensity | Intensity |
| (°) | (A) | (cps) | (%) |
| 4.485 | 19.6865 | 105 | 21.7 |
| 4.715 | 18.7267 | 298 | 61.9 |
| 5.270 | 16.7560 | 168 | 34.9 |
| 5.766 | 15.3161 | 91.6 | 19.0 |
| 7.806 | 11.3165 | 481 | 100 |
| 9.535 | 9.26799 | 383 | 79.7 |
| 10.245 | 8.62703 | 71.9 | 14.9 |
| 11.582 | 7.63438 | 46.4 | 9.6 |
| 12.177 | 7.26235 | 88.5 | 18.4 |
| 14.327 | 6.17731 | 56.5 | 11.7 |
| 16.097 | 5.50159 | 78.7 | 16.3 |
| 16.480 | 5.37470 | 87.6 | 18.2 |
| 16.911 | 5.23878 | 178 | 36.9 |
| 17.083 | 5.18634 | 163 | 33.8 |
| 17.542 | 5.05155 | 108 | 22.5 |
| 17.887 | 4.95502 | 104 | 21.6 |
| 18.243 | 4.85894 | 150 | 31.1 |
| 18.691 | 4.74367 | 144 | 30.0 |
| 19.093 | 4.64470 | 225 | 46.7 |
| 19.437 | 4.56314 | 212 | 44.1 |
| 19.983 | 4.43979 | 160 | 33.3 |
| 20.356 | 4.35922 | 172 | 35.7 |
| 21.562 | 4.11808 | 181 | 37.5 |
| 21.935 | 4.04884 | 101 | 21.1 |
| 22.595 | 3.93198 | 131 | 27.3 |
| 22.887 | 3.88247 | 163 | 33.9 |
| 23.284 | 3.81716 | 126 | 26.1 |
| 24.085 | 3.69203 | 130 | 27.1 |
| 24.597 | 3.61630 | 67.1 | 13.9 |
| 25.297 | 3.51784 | 66.0 | 13.7 |
| 26.224 | 3.39554 | 74.2 | 15.4 |
| 26.846 | 3.31830 | 56.6 | 11.8 |
| 28.797 | 3.09776 | 66.9 | 13.9 |

obtainable by dissolving crude, amorphous or crystalline atorvastatin hemicalcium salt in a protic solvent or optionally in a mixture of protic solvent and apolar or less polar solvent, filtering and cooling the solution to room temperature, optionally seeding said solution with crystals of atorvastatin hemicalcium polymorph B-52 form, optionally stirring the mixture at constant temperature and separating crystalline atorvastatin hemicalcium polymorph form B-52.

15. Medicinal preparations suitable for the reduction of cholesterol-, low density lipoprotein-cholesterol-, apobetalipoprotein- and triglyceride level in blood plasma or treatment of hypercholesterolemia, disbetalipoproteinemia and dislipidemia comprising crystalline atorvastatin hemicalcium salt polymorph B-52 form according to claim 1 and pharmaceutically acceptable vehicles or auxiliary agents.

16. The process for the preparation of medicinal preparations according to claim 15, which comprises admixing crystalline atorvastatin hemicalcium salt polymorph B-52 form with pharmaceutically acceptable vehicles or auxiliary agents and bringing the resulting mixture into galenic form.

17-18. (canceled)

19. The process for the reduction of cholesterol-, low density lipoprotein-cholesterol-, apobetalipoprotein- and triglyceride level of blood plasma or treatment of hypercholesterolemia, dysbetalipoproteinemia and dyslipidemia in a patient in the need of such treatment, which comprises administering said patient a therapeutically effective dose of crystalline atorvastatin hemicalcium salt polymorph B-52 form according to claim 1.
